(19) United States
(12) Patent Application Publication
(10) Pub. No.: US 2007/0219487 A1
(43) Pub. Date: Sep. 20, 2007

(54) Method to Control Ventricular Rate in Atrial Fibrillation Patients

(75) Inventors: Todor N. Mazgalev, Cleveland, OH (US); Youhua Zhang, Clevleand, OH (US); Mark Maciejewski, Edina, MN (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation, Cleveland, OH (US)

(21) Appl. No.: 10/578,823

(22) PCT Filed: Nov. 10, 2004
(86) PCT No.: PCT/US04/37456

§ 371 Date: Jan. 16, 2007

Related U.S. Application Data
(60) Provisional application No. 60/519,082, filed on Nov. 10, 2003.

(51) Int. Cl. A61M 25/00

(52) U.S. Cl. 604/93.01

(57) ABSTRACT

A method is disclosed to control the ventricular rate in a patient's heart, particularly for treating atrial fibrillation or preventing tachyarrhythmia. The patient's atrioventricular nodal area is injected with autologous fibroblast cells and/or bipolymers to create a barrier to slow the electrical conduction from the fibrillating atria to the ventricles.

### CROSS-REFERENCE TO RELATED APPLICATION

This application is based upon co-pending U.S. provisional patent application Ser. No. 60/519,082, filed Nov. 10, 2003, which application is incorporated herein by reference in its entirety.

### FIELD OF THE INVENTION

This invention is directed to the treatment of atrial fibrillation. More particularly, this invention is directed to treating atrial fibrillation through both reducing the incidence of AF and the control of a patient's ventricular rate to prevent ventricular tachyarrhythmias associated with AF.

### BACKGROUND OF THE INVENTION

Atrial fibrillation (AF) is the most common clinically significant cardiac arrhythmia, with an estimated 2.3 million Americans having AF. The prevalence of AF increases with age, from ˜0.1% among adults younger than 55 years to about 9% of those over 80 years of age. Due to an aging population, the number of AF patients is estimated to increase 2.5 times during the next 50 years.

AF is characterized by a rapid and irregular activation of the atria, typically at 400 to 600 pulses per minute in humans. During AF the ventricular rate is no longer under the physiological control of the sinus node. Instead, it is determined by interactions between the rapid atrial firings and the filtering function of the atrioventricular (AV) node. Despite the life saving role of the AV node, without medication AF still results in excessively rapid, irregular ventricular rate. This condition itself can cause severe symptoms, such as palpitation, light-headedness, and syncope. Even worse, long-term tachycardia resulting from the uncontrolled ventricular rate could lead to tachycardia-induced cardiomyopathy. A proper rate or rhythm control becomes essential to avoid development of severe heart failure.

Currently there are two broad strategic treatment options for atrial fibrillation: rhythm control and rate control. For rhythm control, the treatment is directed toward restoring and maintaining the sinus rhythm. Although ideal, sinus rate cannot be restored and maintained in many AF patients. The other alternative is rate control, the intention being to slow ventricular rate while allowing AF to continue. Recent clinical trials have demonstrated that rate control is as good as rhythm control in terms of morbidity and mortality in the AF patients studied. Ventricular rate control is essential in patients with AF that develop ventricular arrhythmias since in most cases ventricular tachycardia (VT) is fatal. Thus, rate control and anticoagulation therapy can be the primary therapy in a majority of AF patients.

The rate control strategy during AF essentially is directed to efforts to utilize and adjust the filtering properties of the AV node, since the AV node is the only normal structure responsible for conducting atrial impulses to the ventricles. Currently drugs such as, digitalis, R-blockers, and calcium channel antagonists are the most commonly used therapy. However, drugs are not effective in some patients and are not well tolerated by others due to side effects. In those drug-refractory patients AV node modification and AV node ablation with pacemaker implantation are currently used to alleviate symptoms.

However, AV node modification, due to its limited success rate, high recurrence, and higher probability of complete AV block, is recommended only when AV node ablation with pacemaker implantation is intended. Currently AV node ablation with pacemaker implantation is the last choice for patients with drug-resistant AF. This strategy destroys the AV node and results in a lifetime pacemaker dependency.

### OBJECTS OF THE INVENTION

It is an object of the invention to treat atrial fibrillation.

It is also an object of the invention to treat atrial fibrillation through the control of a patient's ventricular rate.

It is a further object of the invention to treat atrial fibrillation in a patient by injection of fibroblast cells into the AV nodal area to modify the conduction without creating heart block when the AV node is destroyed.

It is a yet further object of the invention to enhance the filtering role of a patient's AV node by injecting fibroblast cells into the AV nodal area.

It is a yet further object of the invention to treat atrial fibrillation through the modification of the conduction in the AV node to reduce the incidence of atrial fibrillation and to control a patient's ventricular rate to prevent VT.

It is a yet further object of the invention to treat atrial fibrillation in a patient by injection of biopolymers into or around the AV nodal area to modify AV nodal conduction.

It is a yet further object of the invention to enhance the filtering role of a patient's AV node by injecting biopolymers into or around the AV nodal area.

These and other aspects of the invention will become more apparent from the discussion below.

### SUMMARY OF THE INVENTION

According to the invention, the filtering role of the AV node is enhanced without fully destroying the AV nodal condition. This is achieved in a therapeutic application by injecting fibroblast cells or biopolymers into the AV nodal area.

Fibrous tissue serves as a natural insulator with the cardiac conduction system. At the AV node fibrous tissue is intermingled with AV nodal cells, which intermingling is believed to be responsible, at least partially, for normal AV delay. It is believed that the when fibroblast cells or biopolymers are injected into this discrete area, this creates additional delay of AV conduction. Thus, the ventricular rate should be slowed during atrial fibrillation without a complete AV block.

In accordance with the present invention, cultured autologous fibroblast cells or biopolymers are injected to the AV nodal area, either through a catheter-based approach or by direct injection through an endocardial approach. The fibroblast cells delivered or recruited fibroblast cells will grow within the AV nodal area, thus forming more fibrous tissue and creating further delay (or modifying the conduction pathway without creating total conduction block, known as heart block, necessitating a pacemaker) for the AV conduction. This would result in the slowing of the ventricular rate in AF patients.

One embodiment of the invention comprises a system for treating atrial fibrillation in the heart of a patient that includes a cardiac delivery system coupled to a source of material that comprises fibroblast cells (autologous, i.e., the patient's own cells, taken from a skin biopsy) or biopolymers. The cardiac delivery system is adapted to deliver a volume of the material from the source to a location associated with the patient's heart that includes cardiac cells such that the material is adapted to cause conduction delay at the patient's AV node.

According to another embodiment, the material of the source is adapted to be delivered by the cardiac delivery system into an extracellular matrix between cardiac cells at the AV node.

Another embodiment of the invention comprises a system for treating cardiac arrhythmia in a heart of a patient that includes a cardiac delivery system that cooperates with means for treating a cardiac arrhythmia by delivering autologous fibroblast cells or biopolymers into the cardiac tissue structure associated with the arrhythmia.

In another embodiment of the invention the means includes a source of material that includes fibroblast cells or biopolymers and is adapted to form conduction delay when delivered to and around the AV node. The cardiac delivery system is coupled to the source of material and to deliver a volume of the material from the source to the AV node and form conduction delay there.

According to yet another embodiment of the invention, the cardiac delivery system includes means for locating the AV node.

Another embodiment of the invention comprises providing an overall system that includes: a system adapted to locate the AV node; means to prepare material agent that includes fibroblast cells or biopolymers and is adapted to be injected into the AV node and to provide conduction delay at that location; and a delivery catheter that is adapted to deliver the preparation of material agent to the AV node so as to reduce or eliminate arrhythmia.

Another embodiment of the invention comprises a method for assembling a cardiac arrhythmia treatment system that includes: choosing a delivery catheter that is adapted to deliver a preparation of fibroblast cellular material into a patient's AV node; and coupling the delivery catheter with a volume of fibroblast cellular material agent or biopolymers that is adapted to provide substantial insulation against cardiac conduction within the AV nodal area.

A further embodiment of this invention comprises coupling an injector with the delivery catheter that is adapted to inject the volume of fibroblast cellular material or biopolymers to or around the AV node via the delivery catheter.

Another embodiment of the invention concerns a system for treating atrial fibrillation in a patient that includes a cardiac delivery system and a source of material comprising fibroblast cells or biopolymers coupled to the cardiac delivery system. The cardiac delivery system is adapted to deliver fibroblast cells or biopolymers from the source and substantially into the AV nodal area. The fibroblast cells are thus adapted to form at least a partial conduction block at the AV node.

According to another embodiment of the invention, the fibroblasts are autologous. According to one variation of this embodiment, the autologous fibroblasts are derived from a biopsy of a patient's skin, isolated, amplified or cultured, and injected and/or grafted, by techniques known to those skilled in the art. In one further variation of this embodiment, such fibroblasts are removed from the patient and prepared in a manner that is adapted to be delivered to the AV node. A further feature of this variation includes coupling such a preparation to an appropriate delivery catheter.

According to another embodiment of the invention, the fibroblasts or biopolymers are delivered to or around the AV node in a manner adapted to treat atrial fibrillation.

Another embodiment of the invention comprises a method of delivering autologous fibroblasts or biopolymers using a needle injection system.

Other embodiments of the invention contemplate particular delivery systems and methods, such as using percutaneous translumenal delivery approaches, though other more direct surgical approaches may be used in other variations, and in a particular variation transthoracic minimally invasive systems and methods may be used. Delivery may be done intracardiacally via the cardiac chambers, or epicardially, or transvascularly (e.g., via coronary sinus or septal perforators), according to further appropriate device and method variations, respectively.

Further aspects, modes, embodiments, variations, and features of the invention will be brought out in the following portions of the specification, wherein the detailed description is for the purpose of fully disclosing preferred embodiments of the invention without placing limitations thereon.

### DETAILED DESCRIPTION OF THE INVENTION

According to the invention, atrial fibrillation in a patient is treated by injecting fibroblast cells or biopolymers into the patient's AV node. A system for so treating a patient comprises a source of fibroblast cells or biopolymers, means for identifying the location of the AV node, and a delivery system for delivering the fibroblast cells to the AV node.

The fibroblast cells to be used comprise an injectable material that is adapted to cause a conduction delay in cardiac tissue structures generally with fibroblast cells, which in certain regards are illustrative of materials adapted to cause conduction delay without substantially ablating the cardiac tissue. Examples of other such materials include cells, polymers, especially biopolymers, or other fluids or preparations that interfere with intercellular injections, such as impeding communication across or physically separating cellular gap junctions, and in one particular further example an injectable material containing a collagen agent such as collagen, or a precursor or analog or derivative thereof, or one or more precursor materials that may form collagen, including fibrin sealant, alginates, etc.

Preferred aspects of the invention use fibroblasts in place of other cell types such as myoblasts, stem cells, or other cells that provide sufficient gap junctions with cardiac cells to cause conduction delay. With further respect to cell delivery, the fibroblast cells may be cultured from the patient's own cells (i.e., autologous), or may be foreign to the body, such as from a regulated cell culture.

Fibroblasts are a cell of the type considered highly beneficial mode for creating conduction blocks via cell therapy. In one particular beneficial regard, fibroblasts do not undergo a transition stage from proliferating to mature cells as do skeletal myoblasts. Fibroblasts therefore have a more homogeneous excitation pattern as compared to skeletal muscle. Fibroblasts' electrophysiological properties are fairly consistent from one fibroblast to the next, and are believed to be effective for blocking conduction.

The invention according to the highly beneficial embodiments described herein provides systems and methods to treat atrial fibrillation using fibroblast cell transplantation. In one particular highly beneficial embodiment, the fibroblasts are taken from dermal samples of the patient being treated, and are subsequently prepared appropriately, that is, isolated and amplified (e.g., in a culture/preparation kit), and transplanted to or around a patient's AV node.

The invention, therefore, according to one beneficial embodiment uses autologous fibroblasts from the patient's own body and transplants them to the AV node area. Fibroblasts are cells that can survive and multiply in a low oxygen environment and have the ability to block or change/remodel/modify a conduction pathway.

Therefore, according to certain particular embodiments of the present invention, a patient's own fibroblasts are cultured and transplanted into the patient's AV node where they can proliferate and act as a blocking agent to treat atrial fibrillation.

Fibroblasts are a cell line that typically is associated with tissue damage (i.e., skin damage, AMI) and healing of tissue to produce scar tissue. Activation of fibroblasts occurs in response to injury. These events cause a transition of cell types to activated phenotypes having fundamentally differently different biologic function from corresponding quiescent cells in normal tissue. These cellular phenotypes (arising from coordinated gene expression) are regulated by cytokines, growth factors, and downstream nuclear targets. As in wound healing, fibroblasts are directed to the repair and rebuilding of tissue. Quiescent fibroblasts in normal tissue primarily are responsible for steady-state turnover of extracellular matrix, as disclosed, for example, in the literature.

Skin fibroblasts potentiate the migration to prostaglandin (PDGS) and increase collagen accumulation and matrix metalloproteinase (MMP) synthesis, and net collagen accumulation. This formation of collagen matrix coupled with the lack of gap junction proteins in fibroblasts creates the electromechanical isolation from cardiomyocytes. A total lack of electrical conduction has been observed in regions with fibroblast migration in the myocardium of patients with a previous MI. Therefore, fibroblasts are cells that can be utilized (and proliferated) to create electrical insulation and/or reduction of electrical conduction in regions in the heart such as the AV node.

Fibroblasts can be biopsied from many tissues in the body, e.g., lungs, heart, or skin, isolated, and amplified in culture. The cells can then be introduced via injection, graft delivery, or grafting, with a polymeric carrier or backbone, into a region of the heart where there is a need to reduce the conduction, isolate an arrhythmic pathway, or isolate an arrhythmogenic focus in the cardiovascular system.

Fibroblast material useful according to the invention may include one or a combination of the materials above. For example, embodiments of material that include fibroblasts cells may include or recruit endogenous fibroblasts to the area, other materials, such as fluids or other materials or substrates to provide the cells in an overall preparation as a cellular media that is adapted to be injected, such as, in particular, through a delivery lumen of a delivery catheter. In one embodiment material may include fibroblast cells in combination with a biopolymer agent such as fibrin glue agent, which may itself be provided as two precursor materials that are mixed to form fibrin glue that assists in causing a conduction delay or block when delivered with cells to or around the AV node. In another embodiment the biopolymer agent or a combination of two or more biopolymers may be delivered to or around the AV nodal area. Alginate is a common polysaccharide made from seaweed that is another biopolymer that can recruit fibroblast cells and create a non-destructive conduction modifier. Collagen or preparations thereof, including precursors or analogs or derivatives of collagen, is also considered useful in such combinations.

In general, a “polymer” is herein defined as a chain of multiple units or “mers”. Fibrin glue, for example, contains polymerized fibrin monomers, and is further herein considered an illustrative example of a biopolymer since its components are biological. Thrombin in a kit is an initiator or catalyst which enzematically cleaves fibrinogen into fibrin. The monomers can then polymerize into a fibrin gel or glue.

According to another embodiment of the invention, a preparation of fibroblast cells and a second non-living material are both delivered into the AV node to cause conduction delay there. Preferably, the non-living material is adapted to enhance retention of the fibroblast cells being delivered. In another regard, the non-living material is adapted to further contribute to causing the conduction delay. One particular example of a material that provides significant benefit in such combination with fibroblast cellular therapy is fibrin glue.

Notwithstanding the significant benefit of using fibrin glue alone or in combination with fibroblast cell delivery for treating AF, other suitable substitute materials having similarly beneficial effects in such combination are also contemplated, such as other polymers or molecular scaffolds or materials that intervene sufficiently to inter-cellular gap junctions or otherwise impact the extracellular matrix in AV node tissue structures to substantially delay propagation of arrhythmic conduction from propagating. Moreover, collagen or precursors or analogs or derivatives thereof are further considered useful for this purpose, either in addition or in the alternative to fibrin glue.

For the purpose of further illustration, other more specific examples of delivery devices and methods that may be modified according to this disclosure to achieve the various objectives of the present invention are variously disclosed in one or more of U.S. Pat. No. 5,722,403 to McGee et al.; U.S. Pat. No. 5,797,903 to Swanson et al.; U.S. Pat. No. 5,885,278 to Fleishman; U.S. Pat. No. 5,938,660 to Swartz et al.; U.S. Pat. No. 5,971,983 to Lesh; U.S. Pat. No. 6,012,457 to Lesh; U.S. Pat. No. 6,024,740 to Lesh et al.; U.S. Pat. No. 6,071,279 to Whayne et al.; U.S. Pat. No. 6,117,101 to Diederich et al.; U.S. Pat. No. 6,164,283 to Lesh; U.S. Pat. No. 6,214,002 to Fleischman et al.; U.S. Pat. No. 6,241,754 to Swanson et al.; U.S. Pat. No. 6,245,064 to Lesh et al.; U.S. Pat. No. 6,254,599 to Lesh et al.; U.S. Pat. No. 6,305,378 to Lesh; U.S. Pat. No. 6,371,955 to Fuimaono et al.; U.S. Pat. No. 6,383,151 to Diederich et al.; U.S. Pat. No. 6,416,511 to Lesh et al.; U.S. Pat. No. 6,471,697 to Lesh; U.S. Pat. No. 6,500,174 to Maguire et al.; U.S. Pat. No. 6,502,576 to Lesh; U.S. Pat. No. 6,514,249 to Maguire et al.; U.S. Pat. No. 6,522,930 to Schaer et al.; U.S. Pat. No. 6,527,769 to Langberg et al.; and U.S. Pat. No. 6,547,788 to Maguire et al., the disclosures of all of these references being incorporated herein in their entity by reference thereto.

The present invention generally comprises a method for locating the AV node or junction in the heart and injecting pharmacological or biological compounds into the AV node for purposes of enhancing or retarding electrical conduction within the AV node. By way of example, and not of limitation, an imaging modality such as echocardiography is combined with intracardiac electrograms to identify the AV node. The echocardiographs can be transesophageal (TEE), intracardiac (ICE), or transthoracic (TT). Once the AV node is identified, a catheter with an injection needle and infusion port is positioned into the AV node for purposes of direct infusion of biologically active compounds (e.g., cells, genes, or drugs), either to enhance or retard atrioventricular conduction of electrical impulses.

In one embodiment of the invention, a catheter is inserted into a region of tissue adjacent to the AV node in the heart. The catheter typically comprises an extendable hollow needle and a reservoir containing a biological compound that is fluidically coupled to the needle. Echocardiography images of the catheter and AV node are then acquired. Next, intracardiac electrogram signals are acquired from the AV node using the catheter as a probe. Then, the catheter is positioned over the AV node using the echocardiography images in combination with the intracardiac electrogram signals. Once the AV node is located in this manner, the needle is extended into the AV node and the biological compound is infused into the node.

In accordance with the present invention, the AV node is accurately identified by visualizing its anatomical position. The AV node is located within the triangle of Koch and the His bundle penetrates the ventricular septum at the point where the tendon of Todaro and the tricuspid valve annulus come together (apex of the triangle of Koch). This area of the septum containing the AV node lies adjacent to the central fibrous body as the specialized conduction tissue travels to the ventricle. The central fibrous body is a dense fibrous structure which the aortic valve, mitral valve and tricuspid valve meet. The AV node lies posteriorly to the central fibrous body and due to the offsetting of the atrioventricular valves, the specialized muscle appears in closer proximity to the mitral valve than to the tricuspid valve.

By utilization of the basic features of this anatomy which are similar in many species, the AV node can be identified with sufficient resolution to direct an injection needle into the AV node for the administration of biologically active substances. This is accomplished by combining conventional echocardiography, such as transesophageal (TEE), intracardiac (ICE) or transthoracic (TT) echocardiography, with intracardiac electrograms for precise location of the AV node. This combination of imaging, recording of intracardiac electrograms and an injection needle provide the method for the administration of biologically active substances into the AV node. The use of echocardiography in combination with intracardiac electrograms to identify the AV node and the injection of substances into the AV nodal region to improve or enhance AV conduction has never been previously attempted, and yields superior results over conventional locating methods.

According to a preferred method to locate a patient's AV node, in a first step a catheter is inserted into a region of tissue adjacent to the AV node in the heart. Next, echocardiography images of the catheter and AV node are acquired, and then intracardiac electrogram signals are then acquired from the AV node using the catheter as a probe. The catheter is then positioned over the AV node using the echocardiography images in combination with the intracardiac electrogram signals. Once the AV node is located in this manner, a biologically active substance, e.g., one containing fibroblasts and/or biopolymers, is infused into or around the AV node through the catheter.

It will be appreciated that once the AV node is visualized by the imaging modality, the delivery catheter is guided to the AV node. Correct positioning of the catheter is then confirmed by the characteristic intracardiac electrograms of the AV node. The injection needle is then advanced, and the intracardiac electrograms confirmed before the delivery of biologically active substances. It will also be appreciated that the imaging modality combined with intracardiac electrograms only facilitate use of the intravascular delivery system which can take various forms.

The delivery catheter used in the method described above typically comprises an extendable hollow needle and a reservoir containing a biological compound that is fluidically coupled to the needle. The basic components of this delivery system can include a conventional steerable catheter containing a retractable hollow needle and an ICE catheter. The injection catheter, coupled with the ability to measure intracardiac electrograms from either the tip of the delivery catheter or from the injection needle itself, will greatly enhance the ability to accurately delivery substances within the AV conduction axis. Presently, intracardiac electrograms of the AV node are generally measured by a multi-electrode catheter (4 to 8 electrodes) with a spacing between the electrodes of 1 mm to 5 mm. In addition, positioning of the ICE catheter or injection catheter will greatly be facilitated with the development of long sheaths angulated towards the AV conduction axis. It will be appreciated in the context of a delivery system for implementing the method of the present invention that the term “catheter” as used herein generally refers to a probe that also allows for delivery of a biological compound and is not intended as a limitation to a specific type of delivery device.

It will be further appreciated that the delivery catheter could include various forms of energy delivery such as ultrasound, heat, light, etc., to enhance the uptake of the selected compound, in which two sets of bipolar electrodes will be positioned at the tip of the catheter and the tip of the needle. Alternatively, a silver-piezoelectric crystal near the tip of the infusion catheter can be used as a transponder for localizing the catheter tip during imaging. Such a crystal might also be used as a “range finder” to assess the degree of contact of the needle tip with the tissue to be injected. The catheter can be directed toward the atrioventricular valves with the use of a long guiding sheath.

The fibroblast cells and/or biopolymers are delivered in an amount effective to control ventricular rate, treat atrial fibrillation, and/or prevent ventricular tachyarrhythmia. The fibroblast cells can be injected in amounts of from about one million to about one billion cells per injection, for each of one or more injections. The biopolymers are injected in an amount of from about 0.01 ml to about 5 ml per injection, preferably from about 0.1 ml to about 2 ml per injection. It is contemplated that there could be from one to as many as about 40 to about 100 injections, preferably from about 10 to about 75 injections, more preferably from about 20 to about 60 injections, per treatment.

The material comprising fibroblast cells and/or biopolymers can be injected directly into or around a patient's AV node. Preferably the material is injected in multiple injections that form continuous or discontinuous lines about the AV node in the AV nodal area. It is within the scope of the invention that not all the injections in a given treatment would be the same. For example, the amounts of active ingredient may vary, and some injections may comprise fibroblast cells but no biopolymer while others may comprise biopolymer but no fibroblast cells.

The preceding specific embodiments are illustrative of the practice of the invention. It is to be understood, however, that other expedients known to those skilled in the art or disclosed herein, may be employed without departing from the spirit of the invention or the scope of the appended claims.

What is claimed is:

1. A system for controlling ventricular rate in a heart of a patient, comprising:
a cardiac delivery system; and
a source of fibroblast cells and/or a biopolymer coupled to the cardiac delivery system,
wherein the delivery system is adapted to deliver a volume of material from the source and into or around the patient's AV node, and wherein the volume of fibroblast cells and/or a biopolymer when delivered into or around the AV node causes conduction delay and/or modification of conduction pathways.

2. The system of claim 1, wherein the cardiac delivery system further comprises at least one needle cooperating and adapted to fluidly couple the at least one needle to the source of fibroblast cells and/or a biopolymer to deliver the material to or around the AV node via the at least one needle.

3. The system of claim 1, further comprising an injector assembly that is adapted to inject the volume of material via the cardiac delivery system and into or around the AV node.

4. The system of claim 1, wherein the cardiac delivery system comprises
a delivery catheter with an elongated body with a proximal end portion, a distal end portion, and a lumen extending between a proximal port along the proximal end portion and a distal port along the distal end portion; and
a transeptal delivery sheath having an elongate body with proximal end portion, a distal end portion, and a delivery passageway extending between a proximal port along the proximal end portion and a distal port along the distal end portion,
wherein the transeptal delivery sheath is adapted to provide transeptal access into the left atrium of the heart via the delivery passageway, and wherein the delivery catheter is adapted to be delivered through the delivery passageway transeptally into the left atrium to thereby deliver the volume of material to or around the AV node.

5. The system of claim 1, wherein the cardiac delivery system comprises an intracardiac delivery system.

6. The system of claim 1, wherein the cardiac delivery system comprises an endocardial delivery system.

7. The system of claim 1, where the cardiac delivery system comprises a transvascular delivery system that is adapted to deliver the volume of material into or around the AV node through a vessel wall of a vessel associated with the cardiac tissue structure.

8. The system of claim 1, further comprising a kit adapted to prepare autologous cells as the material in an injectable form for delivery with the cardiac delivery system to or around the AV node.

9. The system of claim 1, wherein the cardiac delivery system comprises at least one needle that is adapted to inject the material into or around the region of tissue at or around the AV node.

10. The system of claim 1, wherein the cardiac delivery system comprises a catheter having an elongated body with a proximal end portion, a distal end portion, and at least one lumen extending between a proximal port located along the proximal end portion and a distal port located along the distal end portion wherein the proximal port is adapted to couple to a source that contains at least a part of the material.

11. The system of claim 1 which is useful for treating atrial fibrillation.

12. The system of claim 1 which is useful for preventing ventricular tachyarrhythmia.

13. A method for controlling the ventricular rate in a heart of a patient, which comprises administering an effective amount of a material comprising fibroblast cells and/or a biopolymer to and/or around the patient's AV nodal area.

14. The method of claim 13 which causes conduction delay at the AV node.

15. The method of claim 13 which reduces the incidence of atrial fibrillation.

16. The method of claim 13 which prevents ventricular tachyarrhythmias.

17. The method of claim 13, wherein the material is delivered to or around the AV node in a delivery device having a distal end with an anchor and the delivery device distal end is anchored to or around the AV node as material is delivered.

18. The method of claim 13, material comprising fibroblast cells and/or a biopolymer is delivered to or around the AV node at least in part transeptally across the atrial septum with a transeptal delivery sheath.

19. The method of claim 13, wherein the fibroblast cells are autologous.

20. The method of claim 13, wherein the material comprises one or more biopolymers.

21. The method of claim 20, wherein the biopolymers are selected from the group consisting of fibrin, collagen, alginate, and precursors and/or derivatives thereof, and combinations of two or more thereof.

22. The method of claim 20, wherein the biopolymer recruits fibroblast cells.

23. The method of claim 13, wherein the fibroblast cells and/or a biopolymer are administered in at least one injection.

24. The method of claim 23, wherein there are from about one to about 100 injections.

25. The method of claim 24, wherein there are from about 10 to about 75 injections.

26. The method of claim 25, wherein there are from about 20 to about 60 injections.

27. The method of claim 23 wherein from about one million to about one billion fibroblast cells are administered in each injection.

28. The method of claim 23 wherein from about 0.01 ml to about 5 ml of biopolymer are administered in each injection.

29. The method of claim 28 wherein from about 0.1 to about 2 ml of biopolymer are administered in each injection.

30. The method of claim 23 wherein there are two or more injections and each injection comprises fibroblast cells, a biopolymer, or fibroblast cells in combination with a biopolymer.
